(19) United States
(12) Patent Application Publication
(10) Pub. No.: US 2011/0184279 A1
(43) Pub. Date: Jul. 28, 2011

(54) SELF-CONTAINED DETECTION CAPSULE FOR INSERTION IN A HUMAN OR ANIMAL BODY

(75) Inventor: Marc Massonneau, Tillieres Sur Avre (FR)

(73) Assignee: QUIDD, St Etienne du Rouvray (FR)

(21) Appl. No.: 13/058,942

(22) PCT Filed: Aug. 13, 2009
(86) PCT No.: PCT/FR2009/051589

§ 371 Date: Apr. 13, 2011

(30) Foreign Application Priority Data
Aug. 14, 2008 (FR) 0855586

(51) Int. Cl. A61B 6/00

(52) U.S. Cl. 600/431; 600/476; 600/473

(57) ABSTRACT

The present invention relates to a self-contained detection capsule (1) for insertion into a human or animal bodily canal, the capsule being free of a light source for illuminating the field of view, and comprising:
an optical detector arranged to detect a light emitted by bioluminescence or chemiluminescence, without light excitation of the field of view of the optical detector by the capsule, and
means for recording, processing and/or transmitting data relating to the light detected by the optical detector.

The subject of the present invention is devices and methods involving luminescence reactions on animals or humans.

The present invention applies more particularly to the viewing of light emitted by chemiluminescence or bioluminescence or any other autogenous light emission process.

The term “bioluminescence” denotes the production and the emission of light (visible or nonvisible) resulting from a chemical reaction involving an intracellular or extracellular enzyme, during which the chemical energy is converted into light energy.

The term “chemiluminescence” denotes the production and the emission of light (visible or nonvisible) resulting from a chemical reaction during which the chemical energy is converted into light.

Moreover, the term “fluorescence” denotes the emission of light (visible or nonvisible) resulting from the excitation of a fluorescent entity.

The Infect Immun. article (2006 September; 74(9): 5391-6. “In vivo bioluminescence imaging of the murine pathogen Citrobacter rodentium”, Wiles S, Pickard K M, Peng K, MacDonald T T, Frankel G.) describes an experiment in which mice are force-fed with bioluminescent bacteria expressing luciferase. The light emitted by these bacteria in the gastrointestinal tract was viewed with the IVIS 50 optical imager from the company Xenogen, from outside the body of the mice.

The article “In vivo monitoring of pancreatic β-cells in a transgenic mouse model”, (Smith S T, Zhang H, Clermont A O, Powers A C, Kaufman D B, Purchio A F, West D B. Molecular imaging (2006), 5 (2): 65-75) describes an animal model of a transgenic mouse expressing the luciferase gene regulated by the insulin promoter and the use of the IVIS 100 imager from the Xenogen company for measuring the light emitted by the pancreas of the mouse.

The article “A transgenic mouse model with a luciferase reporter for studying in vivo transcriptional regulation of the human CYP3A4 gene”, (Zhang W, Purchio A F, Chen K, Wu J, Lu L, Coffee R, Contag P R, West D B, Metab Dispos (2003), 31: 1054-1064) describes an animal model of a transgenic mouse expressing the luciferase gene regulated by the CYP3A4 promoter and the use of the IVIS optical imager from the company Xenogen for measuring the light emitted by the liver.

The article “Differentiation and enrichment of hepatocyte-like cells from human embryonic stem cells in vitro and in vivo”, (Duan Y, Catana A, Meng Yn Yamamoto N, he S, Gupta S, Gambhir S S, Zern M A, Stem cells (2007), 25 (12): 3058-6) describes an animal model obtained by injection, into a mouse liver, of human embryonic stem cells infected with a virus expressing luciferase, and the use of an optical imager for measuring the light emitted by the liver of the mouse.

The article “Dissecting tumor maintenance requirements using bioluminescence imaging of cell proliferation in a mouse glioma model”, (Nature Medicine (2004), 10: 1257-1260) describes an animal model of a transgenic mouse which has bioluminous cervical tumors and the use of an optical imager for measuring the light emitted by the cervical tumors of the mouse.

The article “The characterization and hormonal regulation of kidney androgen-regulated protein (KAP)-luciferase transgenic mice”, (Toxicological Sciences (2004), 46: 266-277) describes an animal model of a transgenic mouse expressing the luciferase gene regulated by the Kap promoter and the use of the IVIS 100 optical imager from the company Xenogen for measuring the light emitted in the kidney of the mice.

The article “Noninvasive bioluminescence imaging of normal and spontaneously transformed prostate tissue in mice”, (Lyons S K, Lim E, Clermont A O, Dusich J, Zhu L, Campbell K D, Coffee R J, Grasse D S, Hunter J, purchio T, Jenkins D, Cancer Res (2006) 66(9): 4701) describes an animal model of a transgenic mouse expressing the luciferase gene regulated by the human promoter of the prostate-specific antigen gene, and the use of the IVIS 100-200 optical imager from the company Xenogen for measuring the light emitted in the prostate of the mice.

The use of optical imagers for viewing, from outside the animal's body, the light emitted internally is not entirely satisfactory since the light is absorbed quite rapidly by the tissues, thereby restricting the study to small animals.

Furthermore, the tissues strongly scatter light, which makes it difficult to precisely locate the source.

Moreover, Aposense and ICE-NIRF fluorescent probes have been used to visualize apoptosis, as described in “Molecular imaging of cell death in vivo by a small molecule probe”, (Aloya R, Shirvan A, Grimberg H, Reshef A, Levin G, Kidron D, Cohen A, Ziv I, Apoptosis (2006), 11: 2089-2101) and “A novel method for imaging apoptosis using a caspase-1 near-infrared fluorescent probe”, (Messerli S M, Prabhakar S, Tang Y, Shah K, Cortes M L, Murthy V, Weissleder R, Breakefield X O, Tung C H, Neoplasia (2004), 6 (2): 95-105). Another fluorescent probe, Prosense 680, from the company Visen, targets cathepsin B expressed in many cancers.

These fluorescent probes can generate, by fluorescence, a light that it is known practice to view with an endoscopic capsule.

Thus, the abstract available on the Internet before publication “Gastrointest Endosc. 2008 May 20. Biochromoendoscopy: molecular imaging with capsule endoscopy for detection of adenomas of the GI tract” (Zhang H, Morgan D, Cecil G, Burkholder A, Ramocki N, Scull B, Lund P K) describes the detection of adenomas in the mouse intestine. The Prosense 680 fluorescent probe from the company Visen is used. This probe, which can be activated by the cathepsin B expressed in the adenomas, made it possible to visualize the cancer using capsule endoscopy. The experiments were carried out on dissected intestines.

Many orally absorbed capsules, intended in particular for diagnosing human digestive tract cancers, are moreover known.

The capsule proposed by the company Mediscience Technology® comprises, on its front face, several light-emitting diodes (LEDs) and a CMOS sensor. This capsule also comprises a microcontroller which communicates remotely with a receiver.

The capsule proposed by the company Given Imaging® comprises a camera and it is arranged to communicate, during its transit through the digestive tracts, with a receiver carried by the patient around the waist.

The capsule proposed by the company Olympus® comprises, on its front face, six white LEDs and a CCD sensor and it is arranged to transmit, to a distant monitor, images of the inside of the intestine in order to detect possible abnormalities, such as bleeding, for example.

The company RF System Lab® proposes, under the name Sayaka™, a double-structured capsule comprising a camera and which is intended for viewing the intestines, the internal structure being spun around by means of a system of magnets. The site http://www.popsci.com/how-it-works/article/2008-03/how-it-works-endoscope-camera-pill indicates, regarding the Sayaka™ capsule, that said capsule does not need a motor to move through the intestines, but does require, however, 50 milliwatts to run the camera, lights and processor. Since a battery is too bulky, the camera draws its power through induction charging. A vest worn by the patient contains a coil that continuously transmits power.

Under the name Norika™, this same company RF System Lab® proposes a capsule that has no electrical power source and that comprises four LEDs and one CCD sensor for acquiring images of the inside of the human body. The LEDs and the CCD sensor are powered wirelessly.

The company Smartpill® proposes a capsule intended to be swallowed and comprising a pH sensor, a pressure sensor and a temperature sensor for carrying out measurements inside the digestive tracts.

The company Intelligent Microsystem Center® proposes a capsule comprising one or more LEDs and an image acquisition system comprising a lens and an image sensor.

The publication “A review of low-power wireless sensor microsystems for biomedical capsule diagnosis”, the content of which is incorporated by way of reference, gives a review of wireless communication systems applied to biomedical capsules.

It is also known practice, through International Application WO 2004/032621 to view a canal by means of a capsule introduced into this canal in order to detect whether target cells are present. This capsule comprises a light source that illuminates the canal and a CMOS sensor for detecting the fluorescence emitted by the cells when they are illuminated.

With the capsules mentioned above, the region that it is desired to view is illuminated or excited in a given wavelength range. The light emitted by the source is thus superimposed on the light emitted in response by the targeted organic compounds located in the field of this illumination, thereby reducing the useful signal-to-noise ratio and thus affecting the quality of the view.

In addition, such known capsules are suitable for viewing the surface of a human canal, but not for viewing organs located behind such canals.

Application WO 2004/045374 describes a method in which a radioactive, magnetic, fluorescent or ultrasonic marker, emitting a signal detected by a capsule swallowed by the patient and comprising a detector suitable for the marker used, is introduced into the body.

WO 97/01986 discloses an implantable device for measuring, regulating, monitoring and transmitting blood constituent levels, comprising a sensor which detects, in vivo, the level of at least one blood constituent, present in the vascular tissue of a mammal, and comprises a source of radiation ranging from infrared to visible light. The source is designed so as to direct this radiation toward the tissue where it is modified by its interaction with said tissue. The publication envisions delivering doses of medicaments or modulating an organic function in response to a control signal resulting from the measurement by the sensor.

U.S. Pat. No. 7,033,322 describes a sensor that can be implanted in a blood vessel in order to control an insulin pump.

There is a need to take advantage of effective solutions for studying, screening and treating diseases, inter alia.

### SUMMARY

The invention proposes a novel capsule for insertion into the human or animal body and uses of this capsule for many purposes, diagnostic or therapeutic purposes.

The capsule may be intended to be used for a short period of time in the body, for example the intestinal transit time in the case where the capsule is swallowed.

The capsule may also be left in the body for a longer period of time, for example so as to periodically provide information on a physiological condition of the individual.

The capsule can be inserted into a canal, for example the digestive canal, or into a tissue or organ.

The term “capsule” should not be interpreted in a limited manner and covers any fixed or mobile device which makes it possible to carry out the desired functions after insertion into the body. The term “capsule” can thus cover an implant which is left immobile in the body for a more or less long period of time, or even which is never removed.

Capsule

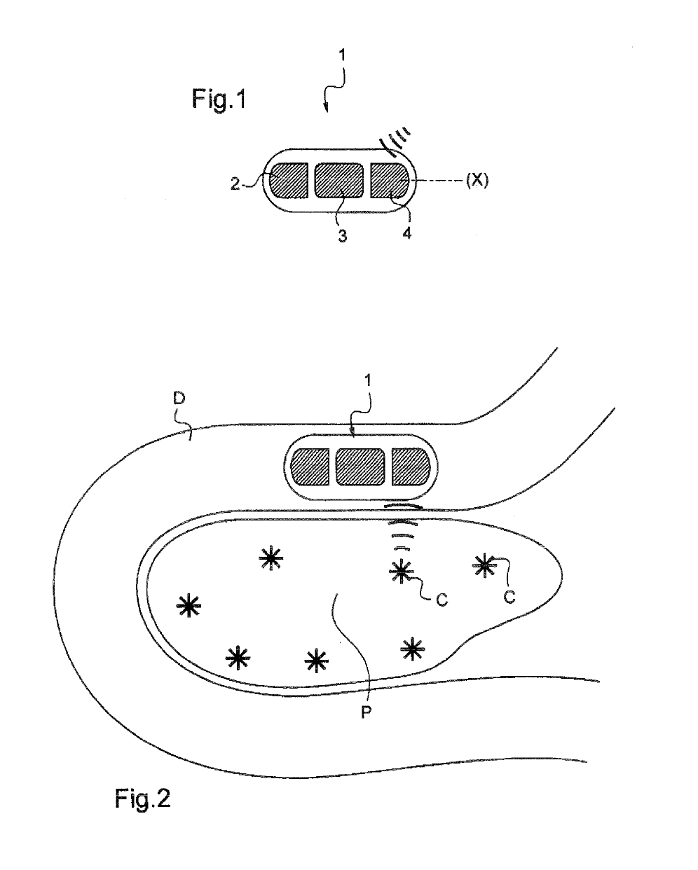

An example of a capsule 1 prepared in accordance with the invention has been represented very diagrammatically in FIG. 1.

The capsule may have various shapes, depending in particular on whether or not it is intended to move. A generally flattened shape may facilitate a static positioning, whereas a shape of revolution may facilitate its transit.

The subject of the invention is in fact, according to a first of its aspects, a self-contained detection capsule for insertion into the human or animal body, in particular into a canal thereof, and which is free of a light source for illuminating the field of view, in particular during the detection, it being possible for the capsule to comprise, as illustrated in FIG. 1:
an optical detector 4 having a field of view and arranged to detect a light emitted by bioluminescence or chemiluminescence, without light excitation of the field of view of the optical detector by the capsule, and
means 3 for recording, processing and/or transmitting data relating to the light detected by the optical detector.

The term “self-contained capsule” denotes a capsule which, when it is inserted into the human or animal body, is not connected to the outside of the body by any wire connection.

The self-contained capsule is for example inert, when it is inserted into the human or animal body. The term “inert” is intended to mean a capsule which does not interact with the human or animal body when it is inserted therein.

The capsule may comprise a power supply means 2.

The term “light source” is intended to mean a source capable of producing a significant radiation in the visible, ultraviolet or near infrared range, used for the detection. It is, for example, an LED or the like.

The absence of light excitation makes it possible to avoid the viewing of the useful signal, corresponding to the light emitted by bioluminescence or chemiluminescence, by the optical detector being disrupted by a prior or simultaneous light excitation of the field of view of the optical detector. The signal/noise ratio is improved thereby. The improvement in the signal-to-noise ratio is capable of simplifying the processing carried out in order to retrieve detection data from the exploitable information and of increasing sensitivity.

In addition, the absence of light source makes it possible to increase the autonomy or, at equivalent autonomy, to reduce the size of an electrical power source placed, where appropriate, on board the capsule, which can make it possible to reduce the bulk of the capsule and, consequently, to facilitate its transit. This may also allow induction powering at a greater distance, since the power required by the capsule may be lower.

The absence of light source also contributes to reducing the bulk of the capsule and may make it possible, for example, at equal bulk, to use an optical detector having a greater sensitivity or a wider field of view. This may also make it possible to have a larger number of sensors on the capsule, in order to obtain better sensitivity and/or better localization of the origin of the detected light, for example about its axis.

Moreover, the detection of light emitted by autogenous luminescence is capable of facilitating the interpretation of the data resulting from the detection. It is no longer necessary to have a specialist look at a video recording taken by a camera on the capsule in order to analyze the detection data, as is the case of certain known capsules, since the capsule can for example, in the case of a diagnostic application, detect or not detect an autogenous luminescence. Only in the event of the detection of an autogenous luminescence will additional examinations possibly be carried out, or will the capsule possibly accomplish one or more predefined actions.

Capsule Casing

According to the intended application, the capsule casing may be made of one or more different materials. For example, in the case where the capsule is swallowed, it is sufficient for the casing not to be toxic and to withstand intestinal transit. When the capsule is intended to be left permanently in the body for several years, the casing should exhibit sufficient biocompatibility.

The external surface of the capsule is, for example, defined by one or more biocompatible materials, such as a resin, for example a polyether-terketone resin.

The capsule may comprise a casing resistant to certain physiological parameters of the digestive canal, in particular to an acidic medium.

The capsule may comprise a manganese dioxide (MnO₂) coating in order to create a laxative effect allowing easier and faster transit in the digestive canal. Such an example of a manganese dioxide coating is disclosed in International Application WO 2004/045374.

The capsule casing should not impair the reception of light by the optical detector, in the case where the detector is located behind the casing. The capsule casing may be made of one or more materials that are transparent to the emission wavelength of the autogenous luminescence, for example transparent to near infrared. The capsule casing is, for example, formed from a plastic or a resin that is molded over the internal components of the capsule, this plastic or resin being both sufficiently biocompatible and sufficiently transparent to near infrared or to another infrared or visible luminescence emission wavelength. Where appropriate, the external surface of the casing can have a shape calculated so as to concentrate the light on one or more sensors of the optical detector, in order, for example, to increase the field of view.

A portion of the external surface of the capsule may be defined by a window of the optical detector, for example one or more lenses of the optical detector. The rest of the external surface may be defined, for example, by a molded material in contact with this or these lens(es), in order to keep them in place and to ensure that the capsule is leaktight.

The capsule should make it possible to recover data resulting from the detection. The capsule casing is produced in such a way as not to impair this data recovery that it performs with contact or without contact.

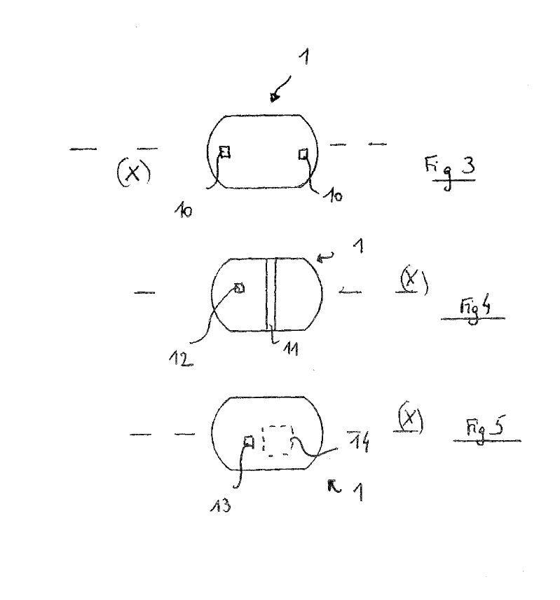

The capsule casing may be completely physiologically inert with respect to the surrounding environment. The capsule casing may further be configured so as to interact with its environment, for example in a programmed or triggered manner, for diagnostic or therapeutic purposes. The capsule casing may, for example, comprise, as represented in FIG. 3, at least two electrodes 10 for carrying out, for example, electrical conductivity measurements or emitting an electric field into the surrounding environment. The capsule may, for example, emit a radio frequency energy for curative purposes, in response to a detection representative of a cancer activity, with a view to destroying said cells. The energy required for the production of the radio frequency field can optionally be obtained by induction, by placing the capsule in an appropriate electromagnetic field.

The capsule casing may also be produced in such a way as to transmit heat to the surrounding environment, for curative purposes. The heat can be produced at the external surface of the capsule or internally to the capsule casing. The capsule may comprise a heat-producing means in order for raising at least one region of the external surface of the capsule to a temperature of greater than or equal to 40° C.

In one exemplary embodiment represented in FIG. 4, the heat-producing means comprises a short-circuit ring 11 which, placed in a fluctuating magnetic field external to the body, receives energy by induction and gives off heat via the Joule effect. The capsule may comprise a temperature sensor 12 and transmit information representative of the surface temperature of the capsule to the external field-emitting device in order to regulate the magnetic field and to keep the temperature at an optimum value for the treatment to be carried out. The short-circuit ring 11 may be a metal ring external to the capsule or internal to said capsule.

The capsule may comprise one or more actuators which make it possible to vary, for example to increase or decrease, its dimensions, once actuated. This can make it possible, for example, to use the capsule to perform a compression of a predefined region in the event of the detection of light representative of a cancer activity. Dilation of the capsule can assist in containing the tumor cells or reduce the blood irrigation thereof while awaiting a surgical procedure or radiotherapy, for example.

The capsule casing may comprise an encapsulated active agent, for example an encapsulated anticancer agent, which can be released, for example, by raising the temperature of the capsule. In this case, the capsule comprises, for example, the abovementioned short-circuit ring 11, thereby making it possible to heat the capsule and to bring about release of the active agent as close as possible to the target to be reached.

As a variant, the capsule may send, to the target to be reached, ultrasonic energy or radiofrequency energy, transported by a beam of ionizing particles, of neutrons, or of γ-rays or X-rays.

Means for Supplying the Capsule with Power

The capsule may comprise an onboard electrical power source. The capacitance of the electrical power source is, for example, between 50 mAh and 400 mAh, it being chosen according to the size and the number of components of the capsule to be supplied and the autonomy desired.

The onboard electrical power source is, for example, a cell, for example a button cell, or a battery, for example of lithium polymer type.

The capsule may also be supplied with electrical power by induction, the capsule comprising a core placed in a variable magnetic field generated outside the body. For example, the patient who has swallowed the capsule can carry, around the waist, a device creating an electromagnetic field that makes it possible to supply the capsule with power.

The capsule may also be powered by a battery/induction hybrid power supply system, by producing the capsule with a battery and with a coil intended to be subjected to an electromagnetic field in order to generate an electric current for recharging the battery. The absence of light source in the capsule can facilitate the implantation of such a hybrid power supply system in the capsule, for a given bulk.

When the capsule comprises a core for generating an electric current that is of use for powering components of the capsule, the inductive coupling between this core and the system generating the electromagnetic field can be modified by the capsule, for example by short-circuiting the core, with a view to transmitting data out of the body in which the capsule is placed.

The capsule may also, where appropriate, be powered by conversion of movement into electricity or by conversion of body heat into electric power.

Optical Detector

The optical detector is, for example, sensitive to visible light (380-780 nm) or nonvisible light.

The optical detector is, for example, sensitive to near infrared. The term “near infrared” denotes radiation of wavelengths between 600 and 900 nm, in particular 650 and 850 nm.

The detector may be arranged to view only in one direction of view or, as a variant, in several directions, in which case the optical detector is termed multi-axis.

The optical detector may be formed from one or a multitude of sensors, for example one or more photodiodes or phototransistors. The optical detector may comprise a plurality of optical detectors arranged to localize the emission of light relative to the capsule.

The optical detector may comprise sensors placed in various ways, at the core of the capsule or at its surface.

The sensors used are, for example, placed according to a convex or hemispherical detection surface.

As a variant, the sensors are placed according to a flat detection surface.

The entire surface of the capsule is, for example, covered with sensors, in particular microsensors.

An appropriate system of lenses and/or reflectors can allow multi-axis viewing while at the same time having a flat detection surface, in order to obtain a field of view of, for example, greater than 180°. The optical detector is, for example, arranged in such a way as to be able to achieve a sensitivity ranging up to the detection of a single photon, by virtue of the presence of a light-amplifying structure within the optical detector. The detector may comprise at least one sensor with optical amplification, for example one or more photocathodes in order to release electrons under the effect of the incident light, it being possible for said electrons to be amplified by an avalanche phenomenon.

The detector comprises, for example, one or more sensors of the EMCCD, ICCD, EBCCD or CMOS type.

The optical detection may comprise, where appropriate, one or more selective filters of bandwidth chosen according to the wavelength of the light produced by the autogenous luminescence.

The selective filter(s) may be obtained, where appropriate, by means of a multilayer deposit formed on the capsule casing.

The optical detector may comprise a matrix of sensors or one or more linear arrays of sensors or a ring of sensors.

Means for Recording, Processing and/or Transmitting Data

The data resulting from the detector may be in the form of an analog or digital signal, depending on whether the detector comprises one or more integrated circuits for implementation of the signals and reading of the sensors.

The capsule may comprise, where appropriate, an analog/digital convertor.

The detection data may, where appropriate, be subjected to analog processing, for example integration processing. For example, the detector comprises a photodiode and the current derived from the photodiode, which is proportional to the amount of light received, can charge a condenser which thus performs the signal integration.

The capsule may comprise a processing unit, for example a microcontroller or microprocessor, for processing and storing the data relating to the light detected by the optical detector.

The capsule may comprise an emitter, for example a radiofrequency emitter, for communicating the data processed by the detector or the processing unit integrated in the capsule to an external receiver located outside the human or animal body in which the detection takes place.

As a variant, the capsule is free of any processing unit and the data relating to the light detected by the optical detector are directly transmitted in real time to an external receiver in which the data processing takes place. The capsule may comprise an electronic memory for recording detection data. Said detection data can be transferred remotely, upon external request. The capsule may comprise a means for automatically triggering detection phases periodically.

The processing carried out by the capsule can consist of an amplification and/or formulation of the signal delivered by the detector, with a view to sending it, to the external receiver, in the form of an analog or digital signal.

The processing may be more complex, and comprise, for example, the integration of the signal delivered by the detector and/or the application of filters and/or various mathematical transforms.

Whether the processing is carried out in the capsule and/or the external receiver and/or in a computer to which the external receiver transmits the data relating to the detection carried out by the detector, the processing may comprise integration of the signal over time, in order to know the overall amount of signals received by the detector.

The capsule may receive information at the time of its activation, which will be able to be used for the processing carried out in the capsule.

The processing may take into account information transmitted by ancillary sensors which do not belong to the optical detector, for example movement or orientation sensors.

The capsule may, as represented in FIG. 5, control at least one onboard actuator 13. The data processing carried out by the capsule can be intended to initiate the actuator 13 automatically or on command, when predefined conditions are met. The actuator 13 is intended, for example, to open a reservoir 14 causing a substance to diffuse out of the capsule, or to dilate the capsule.

Data Transmission

The capsule may comprise a radiofrequency emitter. The latter may be used for real-time transmission of detection data or, as a variant, may be used only after recovery of the capsule, in order to download the content of a memory in which the detection data have been stored.

The transmission of data from the capsule may be carried out in packets, this mode of transmission being more economical in terms of energy.

The radiofrequency emitter emits, for example, in the 20 MHz-40 MHz frequency band, but other frequencies may be used.

When the capsule is recovered after having performed a detection phase in the body, the information relating to the detection may be transmitted to a reader other than by radiofrequency transmission, for example by infrared transmission or by means of an electrical signal carried by an electrical line, after establishment of an electrical link between the capsule and the reader.

The capsule may comprise a data receiver.

These received data relate, for example, to detection parameters, for example the processing to be carried out on the data resulting from the optical detector. The receiver may also receive data which control the turning on of the optical detector, for example in order to measure the emitted light only while the capsule passes in front of a given organ.

The capsule, when it is implanted in the body on a long-term basis, may also receive data concerning the frequency with which the detection should take place.

When the capsule is powered by induction, the electromagnetic induction field may be modulated in order to transmit data to the capsule.

Other Sensors

The capsule may comprise one or more nonoptical sensors.

The capsule may comprise sensors for measuring physiological parameters, for example relating to the canal into which it is inserted, for example a pH, conductivity, temperature or pressure sensor. The capsule may comprise biosensors, for example of DNA chip type.

The capsule may comprise a sensor integrating a light source that remains off during detection of the autogenous luminescence.

Substances Released by the Capsule

It is possible to take advantage of the presence may the capsule in the body for releasing one or more substances. This or these substance(s) may be released for therapeutic or diagnostic purposes. The release can be programmed with respect to time, can be automatic in response to a detection of luminescence and/or can be controlled from outside the body, for example by means of a radiofrequency signal.

The capsule may comprise a compartment containing a substance intended to be released when the capsule is in the human or animal body. This compartment may, for example, communicate with the exterior of the capsule by means of a microvalve.

The capsule may, for example, release a substance that is of use for the detection and that is intended to bind to target cells so as to bring about the emission, by bioluminescence, by said target cells, of light radiation that may be detected by the capsule. Such a substance uses, for example, the renilla luciferase/coelenterazine pairing.

In the example where the light radiation that may be detected by the capsule is brought about by a chemiluminescence reaction, the substance that is of use for the detection is, for example, chosen according to the teaching of International Application WO 2006/129036 in the name of the applicant.

The substance released by the capsule may also have curative properties. It may, for example, be an anticancer agent.

The capsule may be arranged to automatically release a substance contained in the capsule in the event of light detection. As a variant, the capsule may release a substance on command received from the outside. An image of the body containing the capsule is, for example, acquired by medical imaging and, when the capsule is in a given region of the channel, the order to release the substance is transmitted to the capsule.

The capsule may be arranged to release the substance according to predefined conditions detected by nonoptical sensors.

The capsule comprises, for example, a pH-sensitive sensor and the substance is released when the pH value measured reaches a predefined threshold, representative of the presence of the capsule in a given region.

In another variant, the capsule comprises a sensor that detects the presence of a specific enzyme and the substance is released when said enzyme is present in amounts above or below a predefined threshold.

Use of the Capsule as an Assistance to Treatment

The capsule may be of use for improving the precision of a treatment carried out from outside the body carrying the capsule. For example, the capsule may signal a detection of autogenous light, for example representative of a metastatic activity, while it is at a given location along its path in the body. Knowledge of its position at the time of the signaling may be used to focus an ultrasonic beam, a radiofrequency beam, a shock wave or X-rays or γ-rays, of a beam of neutrons, protons or hadrons onto a target, the coordinates of which are precisely known by virtue of the capsule.

The capsule may contain, in one exemplary embodiment of the invention, a material which allows the in situ formation of a radioactive isotope at the level of the capsule, under the effect of high-energy-particle bombardment from outside the body.

Detection Methods

The subject of the invention is also, according to another of the aspects thereof, a method of viewing the human or animal body, comprising the step consisting in:
detecting a light originating from a bioluminescence or chemiluminescence reaction by means of an optical detector inserted into the body.

The method can comprise the prior step consisting in bringing about a bioluminescence or chemiluminescence reaction in said body.

The optical detector may belong to a capsule as defined above.

The capsule is, for example, inserted into a human or animal canal, for example the digestive canal, and makes it possible to view light emitted by the wall of the canal, for example the intestinal wall, or by an organ located behind the wall, for example the pancreas, the liver, the spleen, the kidney or the ovary.

As a variant, the capsule is inserted into a respiratory, vaginal or else arterial canal.

The capsule may also be inserted into the body by being implanted therein on a long-term basis. In this case, the capsule may operate intermittently, during a period of examination of the body which carries it. For example, the capsule is placed in an electromagnetic field which transmits energy to it and allows it to activate the optical detector.

The method may comprise the step consisting in sacrificing the animal, for example after viewing. A part of the wall of the canal or of the organ located behind the canal having emitted the light originating from a bioluminescence of chemiluminescence reaction can then be removed, as appropriate, The capsule may be recovered before or after having sacrificed the animal.

Prostate Treatment

A capsule may be left in permanently in order to periodically detect or detect on command a possible light emission linked to a previously absorbed marker.

The marker is, for example, the PMSA fluorescent marker as described in the publication Humblet et al., 2005 Molecular Imaging.

Dual-Capsule Set

The subject of the invention is also, according to another of the aspects thereof, a set of self-contained capsules, comprising:
a first capsule as defined above,
a second capsule, distinct from the first and arranged to release a substance intended to bring about the emission by chemiluminescence or bioluminescence of light in the presence of a predefined target, it being possible for this light to be detected by the first capsule.

The second capsule is, for example, swallowed before the first and releases, at a predefined location, a substance which will generate a light that may be detected by the first capsule. The substance released by the second capsule may optionally undergo a hepatic pass.

The subject of the invention is also, according to another of aspects thereof, a set of self-contained capsules, for example initially contained in the same packaging, comprising:
a first capsule arranged to detect a light emitted by fluorescence, and comprising, to this effect, a light source for exciting the fluorescence, and
a second capsule arranged to release a fluorescent marker which binds to target cells.

The fluorescent marker is, for example, one of those of the prior art previously commented upon.

The second capsule may comprise a compartment containing the substance to be released and the compartment may open in response to an order received from outside the body, or the opening may result from the detection of certain parameters of the environment of the capsule, by means of at least one sensor present on the second capsule, for example a pH sensor.

It is also possible to optionally use several markers emitting at different wavelengths, in order to carry out a selective detection.

Method of Viewing by Means of a Catheter

The subject of the invention is also, according to another of the aspects thereof, a method of viewing the human or animal body, comprising the step consisting in:
viewing a light originating from a bioluminescence of chemiluminescence reaction by means of a catheter inserted into a canal of said body.

The method may comprise the prior step consisting in bringing about a bioluminescence or chemiluminescence reaction in said body.

The method may be applied to an animal and may be followed by the sacrifice of the animal.

The catheter comprises for example a tube, mounted at the end of which is an optical detector as described above. The diameter of the tube is suitable for its insertion into the region to be explored.

The catheter may also comprise one or more optical fibers for transmitting the light emitted by the body to an optical detector placed outside the body.

Methods of Screening for a Pathological Condition

The subject of the invention is also, according to another of the aspects thereof, a method of screening for a pathological condition in a human or animal body, comprising the steps consisting in:
detecting a light originating from a bioluminescence or chemiluminescence reaction by means of at least one capsule as defined above, and inserted into the body, or of at least one catheter comprising an optical detector, and inserted into the body,
analyzing the light detected, and
evaluating whether the human or animal body is suffering from the pathological condition.

The pathological condition is, for example, diabetes or a cancer of one of the organs of the digestive canal or of an organ located in proximity to the digestive canal.

Two bioluminescence or chemiluminescence reactions emitting at different wavelengths can be brought about by means of two distinct substances, previously introduced into the body, for example two 1,2-dioxetanes bearing different substituents making it possible to regulate the emission wavelengths of several luciferin/luciferase pairings.

The detection, by means of the capsule or by means of the catheter, of the light radiation corresponding to the various wavelengths may make it possible to detect the pathological condition with a lower risk of error.

The subject of the invention is also, according to another of the aspects thereof, a method of viewing a bioluminescence or chemiluminescence reaction in a human or animal body, in which:
a self-contained detection capsule as defined above is inserted into a canal of said body, for example the digestive canal, said capsule comprising:
   an optical detector arranged to detect the light emitted by bioluminescence or chemiluminescence by said body, and
   means for recording, processing and/or treating the data relating to the light detected by the optical detector,
data relating to the light detected by the optical detector of the capsule are received by means of a receiver external to the body.

In this method, an image of the body containing the capsule may be acquired by medical imaging.

The capsule may in particular emit in real time a signal indicating that the optical detector is receiving the light that it is supposed to detect, and at that point, imaging of the body may be carried out, for example, by radiography, X-ray tomography, MRI or echography, in order to localize the capsule and determine more precisely the region where the light is being emitted.

It is, for example, an emission of autogenous light or by fluorescence. In the event of detection representative of the presence or potential presence of a tumor, the capsule may exert a therapeutic or preventive action, either directly, for example by releasing an active agent, or indirectly, for example by heating under the effect of an external electromagnetic field.

Example of Viewing an Organ Through the Wall of a Canal

An example of application of the invention to viewing the pancreas has been represented in FIG. 2.

In this example, the capsule 1 is inserted into the intestines and moves into the duodenum D. As can be seen in FIG. 2, a considerable part of the pancreas P is bordered by the duodenum in humans, but also in many animals.

In this example, the optical detector 4 detects the light emitted by chemiluminescence or bioluminescence by cells C of the pancreas. The amount of light received, which can be determined by the area under the curve for the signal detected by the capsule, is, for example, proportional to the mass of beta-cells of the pancreas, and can provide useful information on diabetes.

Implantation of the Capsule after a Surgical Procedure

A capsule according to the invention may be implanted in the human or animal body in order to perform monitoring and to detect, for example, the efficacy of certain substances in a treatment or to detect the appearance of cancer cells.

In the example of breast cancer, the capsule may be implanted in a sentinel lymph node, and may be activated periodically so as to provide useful information.

The capsule is, for example, as defined above, arranged to detect a light emitted by autogenous luminescence. As a variant, according to this aspect of the invention, the light may also be emitted by exogenous fluorescence, and the capsule may then comprise a light source capable of exciting this fluorescence.

The capsule may be electrically powered by induction when it is necessary to detect the light and may be left dormant in the inactive state outside the periods of detection.

The capsule may be implanted on individuals at risk, over a certain age.

Post-Therapeutic Detection

A capsule according to the invention may be of use for carrying out post-therapeutic detection, and checking, for example, for the absence of cancer cells at the end of treatment. The capsule may be left in place at the end of the treatment, and can be activated periodically in order to check that the disease has not returned.

Variants

The subject of the invention is also, according to another of the aspects thereof, a self-contained detection capsule comprising:
a casing which allows it to be inserted into the human or animal body,
an optical detector with a light-amplifying structure,
means for recording, processing and/or transmitting data relating to the light detected by the optical detector.

The optical detector may be sensitive to near infrared.

The subject of the invention is also, according to another of the aspects thereof, a method of detecting luminescence in a human or animal body, in which a bioluminescence or chemiluminescence is viewed by means of a detector of the capsule above, without optical excitation of the field of view. The method may be applied to animals. Where appropriate, the viewing of the bioluminescence or of the chemiluminescence may be preceded or followed by sacrifice of the animal.

The viewing may be carried out for the purpose of assessing the therapeutic efficacy of a treatment, in particular an oncological treatment.

The capsule may be used to carry out a therapeutic treatment.

An imaging technique may be implemented in order to acquire at least one image of the body at the moment when a luminescence is detected.

The method may comprise the step according to which energy is sent to a region of the body from which the luminescence detected by the capsule originates in order to bring about the destruction of the cells responsible for the luminescence, in particular ultrasonic energy, thermal energy, radiofrequency energy, or energy transported by a beam of ionizing particles, of neutrons, or of γ-rays or X-rays.

The method may comprise the step according to which a toxic or nontoxic substance, toxic or nontoxic molecules, and/or specific markers are sent to a region of the body from which the luminescence detected by the capsule originates in order to bring about the destruction or targeting of the cells or of the tissue area in conjunction with the origin of the luminescence.

This step of sending a substance, molecules and/or markers may optionally follow a step of transmitting data from the capsule to a receiver external to the human or animal body in which the detection is carried out.

The subject of the invention is also, according to another of the aspects thereof, a method of treating the human or animal body, in which:
a self-contained detection capsule is inserted into the body, this capsule comprising at least one optical detector and at least one means for converting energy transmitted by induction into heat,
information signaling the detection of a luminescence is received from the capsule,
the capsule is subjected to a field which makes it possible to raise its temperature, for the purpose of destroying the cells responsible for the luminescence.

The method may be applied to animals and followed by sacrifice of the animal.

The subject of the invention is also, according to another of the aspects thereof, a method for checking the efficacy of a therapeutic treatment, in which:
a self-contained detection capsule containing an optical detector is inserted into the body,
a marker is administered, and
a response of the body to this marker is detected by means of the capsule.

The method may be applied to animals and followed by sacrifice of the animal.

The subject of the invention is also, according to another of the aspects thereof, a method of viewing the pancreas, in which an optical detector is inserted into the digestive canal, this optical detector being sensitive to a chemiluminescent or bioluminescent light emitted by the pancreas and viewed through a wall of the digestive canal by the optical detector.

The method may be applied to animals and followed by sacrifice of the animal.

The subject of the invention is also, according to another of the aspects thereof, a method of detecting the appearance of cancer cells, in which a self-contained detection capsule is inserted into the body, the capsule comprising a detector sensitive to a phenomenon linked to the appearance of cancer cells,

in which method the detector is periodically activated in order to detect the presence of said phenomenon.

Said phenomenon may be a luminescence brought about by the use of a marker. The luminescence may be exogenous or autogenous.

The detector may comprise a biosensor.

The detector may comprise a sensor with an optical amplifier.

The detector may comprise a microcamera.

The analysis of the data originating from the detector can be carried out outside the body.

Of course, the invention is not limited to the examples which have just been described.

The expression “comprising a” should be understood to be synonymous with “comprising at least one”.

1. A self-contained detection capsule configured to be inserted into the human or animal body, the capsule being free of a light source for illuminating the field of view, and comprising:
an optical detector configured to detect a light emitted by bioluminescence or chemiluminescence, without light excitation of the field of view of the optical detector by the capsule, and
means for at least one of recording, processing and transmitting data relating to the light detected by the optical detector.

2. The capsule as claimed in claim 1, the optical detector being configured to detect near infrared radiation.

3. The capsule as claimed in claim 1, the optical detector being configured to view a field of approximately 180° about an axis of the capsule.

4. The capsule as claimed in claim 1, the optical detector comprising a plurality of sensors covering the entire surface of the capsule.

5. The capsule as claimed in claim 1, the optical detector comprising at least one sensor with optical amplification.

6. The capsule as claimed in claim 1, comprising a radiofrequency emitter for communicating with an external receiver located outside the human or animal body in which the detection is carried out.

7. The capsule as claimed in claim 1, which allows the release of a substance contained in a compartment of the capsule.

8. The capsule as claimed in claim 1, the optical detector comprising a plurality of optical sensors configured to localize the emission of light relative to the capsule.

9. The capsule as claimed in claim 1, comprising a heat-producing means for raising at least one region of the external surface of the capsule to a temperature of greater than or equal to 40° C.

10. The capsule as claimed in claim 9, the heat-producing means comprising a short-circuit ring.

11. The capsule as claimed in claim 1, comprising an actuator.

12. The capsule as claimed in claim 11, the actuator increasing an external dimension of the capsule when actuated.

13. The capsule as claimed in claim 1, which has a flattened shape.

14. The capsule as claimed in claim 1, comprising an electronic memory in which detection data are recorded, an emitter for transferring the data remotely, upon external request, and means for automatically triggering detection phases periodically.

15. A set of self-contained capsules, comprising:
a first capsule as claimed in claim 1, and
a second capsule, distinct from the first and configured to release a substance intended to bring about the emission by chemiluminescence or bioluminescence of light in the presence of a predefined target, it being possible for this light to be detected by the first capsule.

16. A method of viewing the human or animal body, comprising the steps consisting in:
bringing about a bioluminescence or chemiluminescence reaction in said body, and
detecting a light originating from the bioluminescence or chemiluminescence reaction by means of a capsule as defined in claim 1, inserted into the body, or by means of a catheter inserted into the body and transmitting the light to an optical detector or carrying an optical detector at the end.

17. The method as claimed in claim 16, in which the capsule or the catheter is inserted into a canal, the wall of which is viewed, and in which the light detected is emitted from said canal.

18. The method as claimed in claim 16, in which the capsule or the catheter is inserted into a canal, and in which the light detected is emitted from an organ located behind the canal.

19. The method as claimed in claim 16, the capsule being implanted in the body, and being able to be activated periodically in order to detect a light emitted by chemiluminescence or bioluminescence.

20. A self-contained detection capsule comprising:
a casing which allows it to be inserted into the human or animal body,
an optical detector with a light-amplifying structure,
means for at least one of recording, processing and transmitting data relating to the light detected by the optical detector.

21. The capsule as claimed in claim 20, the optical detector being sensitive to near infrared.

22. A method of detecting luminescence in a human or animal body, in which a bioluminescence or chemiluminescence is viewed by means of a detector of the capsule as claimed in claim 20, without optical excitation of the field of view.

23-29. (canceled)

30. A method of treating the human or animal body, in which:
a self-contained detection capsule is inserted into the body, this capsule comprising at least one optical detector and at least one means for converting energy transmitted by induction into heat,
information signaling the detection of a luminescence is received from the capsule,
the capsule is subjected to afield which makes it possible to raise its temperature, for the purpose of destroying the cells responsible for the luminescence.

31. (canceled)

32. A method for checking the efficacy of a therapeutic treatment, in which:
a self-contained detection capsule comprising an optical detector is inserted into the body,
a marker is administered,
a response of the body to this marker is detected by means of the capsule.

33. (canceled)

34. A method of viewing the human or animal body, comprising the step consisting in:
detecting a light originating from a bioluminescence or chemiluminescence reaction by means of an optical detector inserted into the body.

35-41. (canceled)

42. A set of self-contained capsules, comprising:
a first capsule configured to detect a light emitted by fluorescence, and comprising, to this effect, a light source for exciting the fluorescence, and
a second capsule configured to release a fluorescent marker which binds to target cells.

43. A method of viewing the human or animal body, comprising the step consisting in:
viewing a light originating from a bioluminescence or chemiluminescence reaction by means of a catheter inserted into a canal of said body.

44. (canceled)

45. A method of screening for a pathological condition in a human or animal body, comprising the steps consisting in:
detecting a light originating from a bioluminescence or chemiluminescence reaction by means of at least one capsule according to claim 1, and inserted into the body, or of at least one catheter comprising an optical detector, and inserted into the body,
analyzing the light detected, and
evaluating whether the human or animal body is suffering from the pathological condition.

46. A method of viewing the pancreas, in which an optical detector is inserted into the digestive canal, this optical detector being sensitive to a chemiluminescent or bioluminescent light emitted by the pancreas and viewed through a wall of the digestive canal by the optical detector.

47. (canceled)

48. A method of detecting the appearance of cancer cells, in which a self-contained detection capsule is inserted into the body, the capsule comprising a detector sensitive to a phenomenon linked to the appearance of cancer cells,
in which method the detector is periodically activated in order to detect the presence of said phenomenon.

49-55. (canceled)
